(19) United States
(12) Patent Application Publication
(10) Pub. No.: US 2008/0015642 A1
(43) Pub. Date: Jan. 17, 2008

(54) Method for stimulation of the vagus nerve

(75) Inventors: Kristin D. Johnson, Louisville, CO (US); Ronald J. Podhajsky, Boulder, CO (US)

Correspondence Address: COVIDIEN, 60 MIDDLETOWN AVENUE, NORTH HAVEN, CT, 06473, US

(73) Assignee: SHERWOOD SERVICES AG

(21) Appl. No.: 11/487,918

(22) Filed: Jul. 17, 2006

(51) Int. Cl. A61N 1/00

(52) U.S. Cl. 607/2

(57) ABSTRACT

The present disclosure relates to EBS methods for stimulation of the vagus or vagal nerve for weight loss and the like. According to one aspect of the present disclosure, the method of weight loss includes the steps of percutaneously placing at least one EBS needle at a predetermined location of the body in close proximity to the vagus nerve; and energizing at least a distal tip of each EBS needle to treat the vagus nerve at the location of the distal tip of the EBS needle.

### BACKGROUND

1. Technical Field

The present disclosure relates to surgical methods and, more particularly, to energy-based methods for stimulation of the vagus or vagal nerve for weight loss and the like.

2. Background of Related Art

In the United States, excess weight or obesity is a problem for a majority of the population. A 1999 National Health and Nutrition Examination Survey concluded that 61% of American adults are overweight or obese and the percentage of people becoming overweight continues to rise. People who are overweight or obese are generally more prone to ailments such as high-blood pressure, heart disease, and diabetes.

In order to lose weight and combat the conditions associated with excess weight, many individuals partake in numerous methods and/or procedures to lose weight. For example, individuals attempt diets, undertake exercise routines or regimens, purchase and use weight reduction equipment or weight reduction drugs, and the like, or undergo surgical procedures.

Surgery for obesity has included liposuction, gastroplasty and gastric bypass procedures. Gastroplasty, which is also known as stomach stapling, involves constructing a 15 to 30 mL pouch along the lesser curvature of the stomach. A modification of this procedure involves the use of an adjustable band that wraps around the proximal stomach to create a small pouch.

Referring to FIGS. 1 and 2, it is known that the vagus nerve “V” plays an important role in mediating afferent information from the stomach to the satiety center in the brain. The vagus nerve “V” arises directly from the brain but, unlike the other cranial nerves, extends beyond the head. At its furthest extension the vagus nerve “V” reaches the lower parts of the intestines.

The vagus nerve “V” is the dominant nerve of the gastrointestinal (GI) tract, the right and left branches or nerve afferents “V1, V2”, respectively, of the vagus nerve “V” connect the GI tract to the brain. After leaving the spinal cord, the vagal afferents transport information regarding that tract to the brain of the patient “P”. In the lower part of the chest, the left vagus “V2” rotates, becomes the anterior vagus, and innervates the stomach “S”. The right vagus “V1” rotates to become the posterior vagus, which branches into the celiac division and innervates the duodenum and proximal intestinal tract.

It has been discovered that the afferent vagal fibers from the stomach wall increase their firing rate when the stomach is filled, thus sending satiety signals to the brain. These satiety signals cause the individual to either terminate the meal and/or inhibit further feeding for some time afterward. During this postabsorptive (fasting) period, the satiety signals slowly dissipate until the drive to eat again takes over.

A method of treating patients with obesity by bilateral stimulation of the patient's vagus nerve, in which a stimulating electrical signal is applied to one or both branches of the vagus, is disclosed in U.S. Pat. No. 6,587,719, issued Jul. 1, 2003. Generally, vagus nerve stimulation procedures have adopted cardiac pacemaking technology in which a “cardiac pacemaker-like” device is permanently implanted in the patient and connected specific locations of the vagus nerve in order to electrically effect and/or stimulate those specific locations.

### SUMMARY

The present disclosure relates to energy-based methods for stimulation of the vagus or vagal nerve for weight loss and the like.

According to an aspect of the present disclosure, a method for performing a treatment on a human body includes the steps of placing a distal tip of at least one energy-based stimulation (EBS) needle proximal to the vagus nerve of the body; and energizing each EBS needle such that the distal tip thereof treats the vagus nerve.

According to another aspect of the present disclosure, a method of weight loss involving EBS of the vagus nerve is provided. The method includes the steps of percutaneously placing at least one EBS needle at a predetermined location of the body proximal to the vagus nerve; and energizing at least a distal tip of each EBS needle to treat the vagus nerve at the location of the distal tip of the EBS needle.

The method may further include the step of placing a distal tip of a plurality of EBS needles at predetermined locations along a length of the vagus nerve. The method may further include the step of connecting each EBS needle to an EBS generator. The method may still further include the step of monitoring a temperature of the vagus nerve at each location of EBS needle placement.

The method may include the step of energizing each EBS needle with an RF waveform. The RF waveform may have a frequency above about 50 kHz. Each EBS needle may be energized with one of a continuous wave and a pulsed wave.

The method further includes the step of maintaining a temperature of the vagus nerve at each location of EBS needle placement below a threshold level, wherein the threshold level is 42° C.

The EBS needles may be placed at least at a location adjacent the vagus nerve where the vagus nerve innervates the stomach.

### BRIEF DESCRIPTION OF THE DRAWINGS

Further features and advantages of the invention will become readily apparent from the following specification and from the drawings, in which:

FIG. 1 is a schematic illustration depicting vagal nerves in a patient;

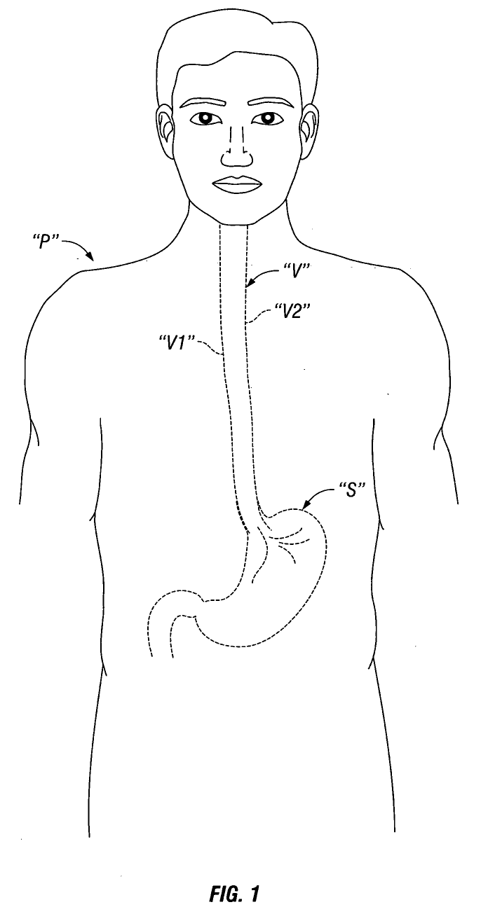

FIG. 2 is an illustration depicting vagal nerve innervations to the visceral organs;

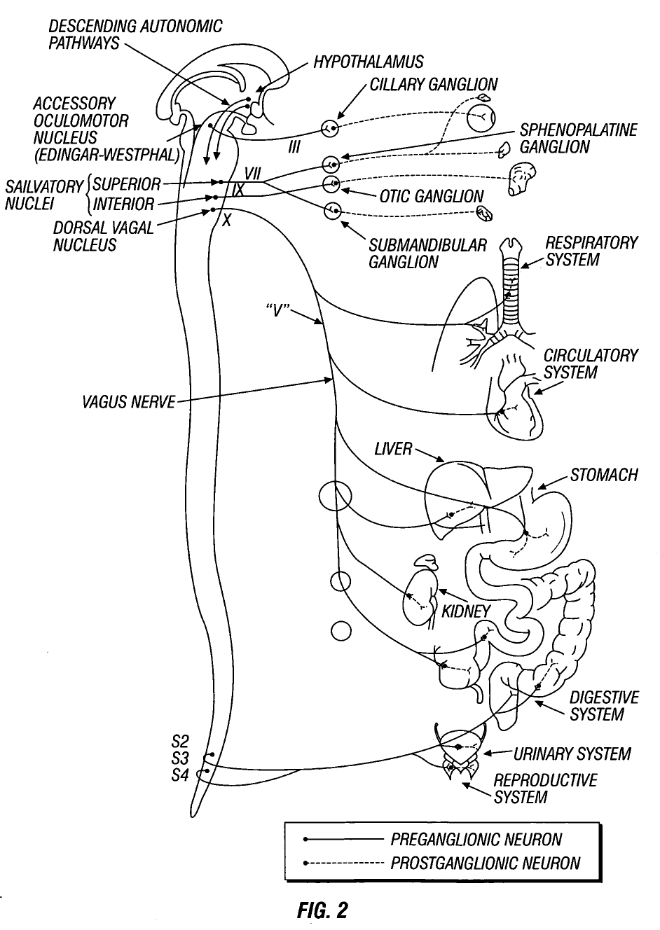

FIG. 3 is a schematic illustration of an EBS system according to an aspect of the present disclosure;

FIG. 4 is a schematic illustration of the EBS system of FIG. 3 shown operatively placed within a patient for energy-based stimulation of the vagal nerve; and

FIG. 5 is a schematic illustration of the EBS system of FIG. 3 shown operatively placed within the patient for energy-based stimulation of the vagal nerve, according to another aspect of the present disclosure.

### DETAILED DESCRIPTION

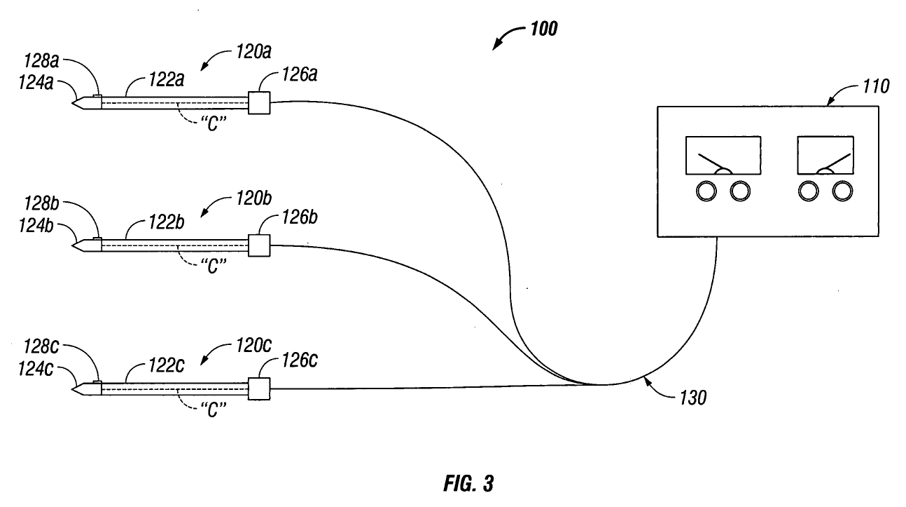

As seen in FIG. 3, an energy-based system (EBS) according to an embodiment of the present disclosure is generally designated as 100. EBS 100 includes an energy-based generator 110, at least one EBS acupuncture needle 120a-120c connectable to generator 110, and an electrical conduit 130 interconnecting each EBS needle 120a-120c to EBS generator 110.

EBS generator 110 is operable to generate and deliver any suitable therapeutic energy-based stimulus to each EBS needle 120a-120c in order to deliver this energy-based stimulus to a target tissue, which may produce weight loss effects. In one embodiment, the therapeutic energy-based stimulation may include a stimulating RF waveform having frequencies above approximately 50 kHz. In some embodiments, the stimulating RF waveform may include continuous or pulsed wave applications at any duty cycle. For example, the pulsed wave application may produce bursts of energy at a rate of approximately 2 bursts per second (i.e., 2 Hz of 20 msec duration each). Also, the tip of each EBS needle 120a-120c is to be maintained at a temperature level that is less than or equal to 42° C. for a period of 120 sec. In some embodiments, the energy-based stimulus may include, for example, pulsed lasers through fiber optics, ultrasonic, piezoelectric, or microwave.

With continued reference to FIG. 3, each EBS needle 120a-120c includes a respective needle probe 122a-122c extending or extendable from a respective handle or hub 126a-126c. Each needle probe 122a-122c may be solid or hollow and may be fabricated from a suitable electrically conductive, rigid material, such as stainless steel. If needle probe 122a-122c is hollow, a cooling fluid may be circulated through a hollow cavity thereof in any suitable manner. In addition, an additional lumen in the hollow cavity may allow for the introduction of any suitable local anesthetic. An exterior surface of each needle probe 122a-122c may be coated with a suitable friction reducing material, such as TEFLON (PTFE) to aid in the percutaneous insertion of each needle probe 122a-122c into the body. Additionally, each needle probe 122a-122c may be coated with a suitable electrically insulative material along substantially an entire length thereof.

A distal tip 124a-124c of each needle probe 122a-122c may be exposed for transmission of an energy-based stimulus therefrom. Each distal tip 124a-124c may be sharpened in order to facilitate percutaneous tissue penetration and precise energy application, or may have an extended surface exposure to provide a broader area of effect in areas where the vagus nerve has a broad innervation field, such as around the stomach and/or intestines.

As seen in FIG. 3, electrical conduit 130 may be connected to each needle probe 122a-122c via hubs 126a-126c. Alternatively, an end of electrical conduit 130 may be selectively electrically connected to at least one needle probe 122a-122c either prior to or after insertion of needle probe 122a-122c into the body. A clip (e.g., an alligator clip or other suitable connector) may be used to connect the end of electrical conduit 130 to a proximal end of needle probe 122a-122c.

Each EBS needle 120a-120c may include a respective temperature sensor 128a-128c disposed at a respective distal tip 124a-124c thereof. Each temperature sensor 128a-128c is electrically connected to EBS generator 110. Temperature sensors 128a-128c may form a component of a feedback control loop “C” capable of automatically adjusting an energy output of EBS generator 110. The energy output of EBS generator 110 may be adjusted so that the temperature of the body tissue in the proximity of distal tips 124a-124c of EBS needles 120a-120c does not exceed a threshold level that may result in local tissue damage. The tip of each EBS needle 120a-120c is to be maintained at a temperature level that is less than or equal to 42° C. for a period of 120 sec.

In accordance with some embodiments of the present disclosure, EBS system 100 may be used to treat various ailments, including and not limited to weight management and/or weight loss. In operation, at least one EBS needle 120a-120c is placed at a specific treatment location on the human body, which specific treatment location coincides with known acupuncture meridians, and stimulated with radio frequency energy.

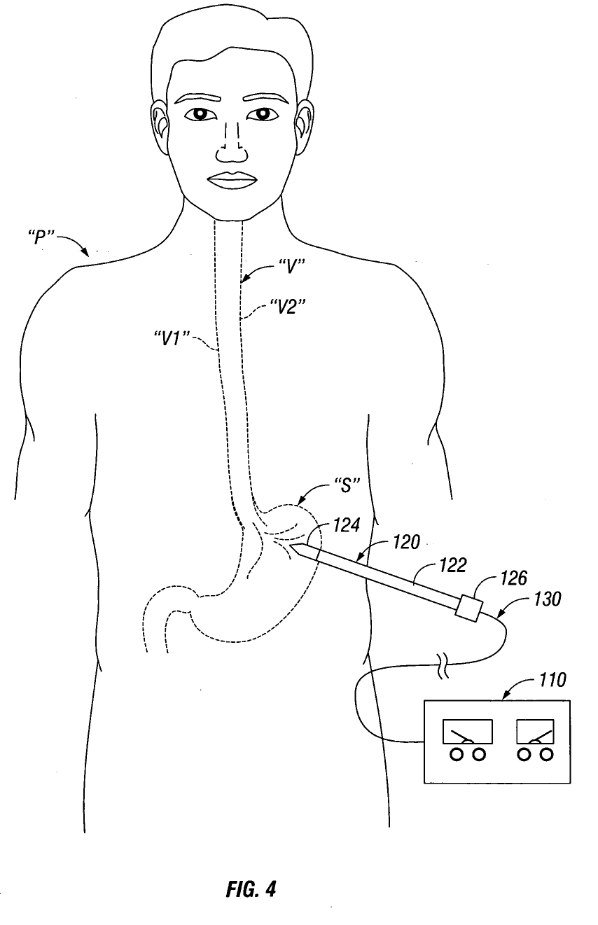

In one embodiment, as seen in FIG. 4, treatment of the vagus nerve “V” or at least one of the right and left branches or nerve afferents “V1, V2”, respectively, may include percutaneous insertion and placement of at least one EBS needle 120a-120c adjacent the vagus nerve “V”. A distal tip 124 of at least one EBS needle 120 may be placed in close proximity to or adjacent to the vagus nerve “V” at a location where the vagus nerve “V” innervates the stomach “S”. With distal tip 124 of each EBS needle 120 placed in close proximity to the vagus nerve “V”, at a location adjacent the stomach “S”, EBS generator 110 is activated to transmit EBS energy to distal tip 124 to treat the vagus nerve “V” at said location.

In an embodiment where RF energy is utilized, each EBS needle 120 may be stimulated with an RF waveform including frequencies above about 50kHz and may include either continuous wave or pulsed wave application at any duty cycle. During application of the RF energy to each EBS needle 120, sensors 128 may monitor the temperature at each insertion site and provide feedback, via control loop “C”, to EBS generator 110. EBS generator 110 may be configured to adjust the parameters of the RF energy transmitted to each EBS needle 120 in response to the feedback from sensors 128. EBS generator 110 may adjust the parameters of the RF energy being transmitted so that the temperature of the vagus nerve “V” does not exceed a threshold that may result in local tissue damage. For example, the threshold temperature for the vagus nerve “V” may not exceed about 42° C.

Additional branches of the vagus nerve “V” may be energy-based stimulated and/or treated, including and not limited to the superior and recurrent laryngeal nerves, the meningeal nerves, the auricular nerves, the pharyngeal nerves, the cardiac nerves, the bronchial nerves, the gastric nerves, the hepatic nerves, the celiac nerves, and the renal rami, pharyngeal, pulmonary, and esophageal plexus, and the anterior and posterior trunks. The auricular branch provides accessible access to the vagus nerve “V” due to its location in the ear lobe.

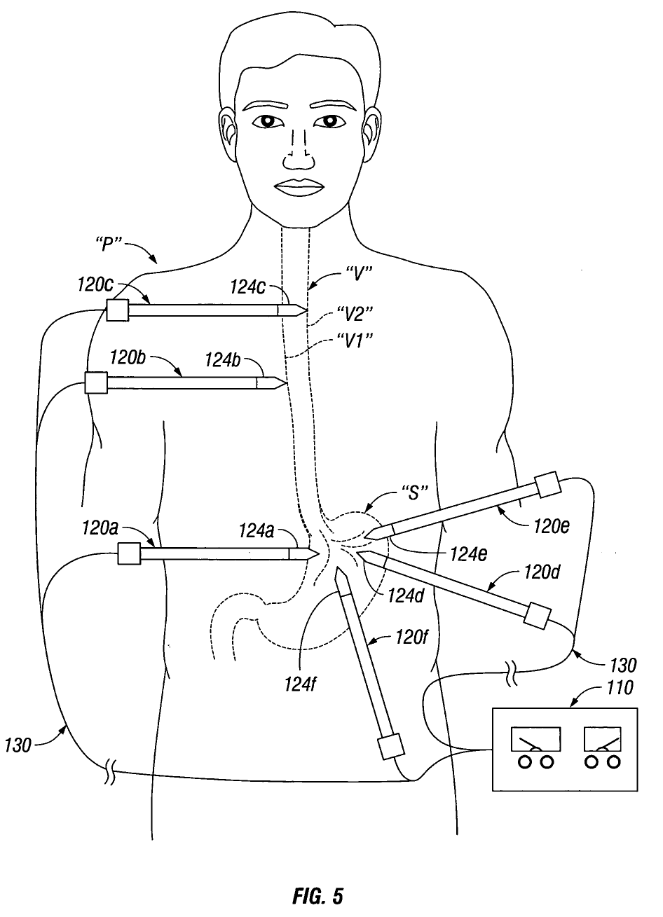

While a single EBS needle 120 has been shown in FIG. 4 as being inserted and/or placed in close proximity to the vagus nerve “V” it is envisioned and within the scope of the present disclosure, as seen in FIG. 5, that a plurality of EBS needles 120a-120c may be inserted into patient “P”, such that respective distal tips 124a-124c thereof are placed at difference locations along the length of the vagus nerve “V” and/or a plurality of EBS needles 120d-120f may be inserted into patient “P”, such that respective distal tips 124d-124f thereof are placed in close proximity to a single location along the length of the vagus nerve “V”.

The foregoing description is merely a disclosure of particular embodiments and is no way intended to limit the scope of the invention. Other possible modifications are apparent to those skilled in the art and all modifications are to be defined by the following claims.

What is claimed is:

1. A method for performing a treatment on a human body, the method comprising the steps of:
placing a distal tip of at least one EBS needle proximal to the vagus nerve of the body; and
energizing each EBS needle.

2. The method according to claim 1, wherein the placing step comprises placing a plurality of EBS needles at predetermined locations along a length of the vagus nerve.

3. The method according to claim 1, further comprising the step of connecting each EBS needle to an EBS generator.

4. The method according to claim 1, further comprising the step of monitoring a temperature of the vagus nerve at each location of EBS needle placement.

5. The method according to claim 1, wherein the energizing step comprises energizing each EBS needle with an RF waveform.

6. The method according to claim 5, wherein the RF waveform has a frequency above about 50 kHz.

7. The method according to claim 1, wherein the energizing step comprises energizing each EBS needle with one of a continuous wave and a pulsed wave.

8. The method according to claim 4, further comprising the step of maintaining a temperature of the vagus nerve at each location of EBS needle placement below a threshold level.

9. The method according to claim 1, wherein the threshold level is 42° C.

10. The method according to claim 1, wherein the EBS needles are placed at least at a location proximal the vagus nerve where the vagus nerve innervates the stomach.

11. A method of weight loss involving EBS of the vagus nerve, the method comprising the steps of:
percutaneously placing at least one EBS needle at a predetermined location of the body proximal to the vagus nerve; and
energizing at least a distal tip of each EBS needle.

12. The method according to claim 11, further comprising the step of connecting each EBS needle to an EBS generator.

13. The method according to claim 11, further comprising the step of monitoring a temperature of the vagus nerve at each location of EBS needle placement.

14. The method according to claim 11, wherein the energizing step somprises energizing each EBS needle with an RF waveform.

15. The method according to claim 14, wherein the RF waveform has a frequency above about 50 kHz.

16. The method according to claim 11, wherein the energizing step comprises energizing each EBS needle with one of a continuous wave and a pulsed wave.

17. The method according to claim 11, further comprising the step of maintaining a temperature of the vagus nerve at each location of EBS needle placement below a threshold level.

18. The method according to claim 11, wherein the threshold level is 42° C.

19. The method according to claim 11, wherein the EBS needles are placed at least at a location proximal the vagus nerve where the vagus nerve innervates the stomach.
